(19) United States
(12) Patent Application Publication
(10) Pub. No.: US 2010/0211118 A1
(43) Pub. Date: Aug. 19, 2010

(54) BONE SCREW AND METHOD OF MANUFACTURING SAME

(75) Inventors: Alexis Christen, Bern (CH); Zeljko Markovic, Selzach (CH)

Correspondence Address: LERNER, DAVID, LITTENBERG,;KRUMHOLZ & MENTLIK, 600 SOUTH AVENUE WEST, WESTFIELD, NJ, 07090, US

(73) Assignee: STRYKER TRAUMA AG, Selzach (CH)

(21) Appl. No.: 12/704,795

(22) Filed: Feb. 12, 2010

(30) Foreign Application Priority Data
Feb. 16, 2009 (EP) 09 002 104.9

(51) Int. Cl. A61B 17/86; B21H 3/02

(52) U.S. Cl. 606/312; 470/10

(57) ABSTRACT

The invention relates to a self-tapping bone screw, in particular for use as a compression screw or a locking screw for an implant. The bone screw has screw shank, which has a front tip, a cutting region, an intermediate region and a rear head region. In a transition region comprising mutually adjoining parts of the cutting region and the intermediate region the root diameter of the screw shank in the cutting region is greater than the root diameter of the screw shank in the intermediate region, and the outside diameter of the screw shank is constant.

### CROSS REFERENCE TO RELATED APPLICATIONS

The present application claims the benefit of European Patent Application No. 09 002 104.9, filed in the European Patent Office on Feb. 16, 2009, entitled “Bone screw and method of manufacturing same”, the disclosure of which is hereby incorporated herein by reference.

### BACKGROUND OF THE INVENTION

The disclosure relates to a self-tapping bone screw, in particular for use as a compression screw or a locking screw for an implant. The disclosure further relates to a manufacturing method for such a bone screw.

Bone screws are screws which are screwed into bones. Basically, bone screws are used in two different ways: In a first application bone screws serve to fix bones or bone fragments in a desired position relative to one another. In this case, the bone screw is used alone. In a second application the bone screw is used as a compression screw or a locking screw in order to position additional elements as fixation elements in or on the bone. Here, bone screws are used, for example, together with marrow nails. Another area of application is osteosynthesis, in which a biocompatible element substitutes for a bone or a bone fragment. For example, a plate made of titanium can be anchored by bone screws to the skull, as a replacement for a skull fragment.

Bone screws are available in a large number of variations for special applications. Thus, for example, U.S. Pat. No. 6,030,162 discloses a bone screw for generating an axial compression, so that bone fragments are pressed together by the screwed-in screw. The compression is generated, inter alia, by providing a plurality of threaded portions having different thread pitches.

In many cases, the screw shank of a bone screw is cylindrically shaped. From EP 0 491 211 A1 there is known a bone screw which has a head-side, cylindrical first shank portion and a tip-side second and likewise cylindrical shank portion adjoining the first shank portion, the first shank portion having a greater root (or core) diameter than the second shank portion. In yet other cases, bone screws have a conically shaped screw shank widening from the tip towards the head.

From WO 2007/048267 A1 there is known a bone screw in which a root diameter in a pre-forming region located at the tip of the screw is greater than the root diameter in an intermediate region adjoining the pre-forming region. An outside diameter of the screw in the pre-forming region is likewise greater than an outside diameter in the intermediate region.

Self-tapping screws have the advantage that a thread does not have to be pre-cut in the bone. Such screws have a screw shank with at least one threaded portion. The thread is suitably configured with respect to properties such as thread profile, flank angle, etc., so that the screw cuts its thread itself when the surgeon screws it into the bone material.

A fundamental problem with self-tapping bone screws are the, in some cases, considerable screwing-in forces which arise when screwing the screw into the bone. The material of a bone behaves to a certain extent elastically when being cut through; that is to say the bone material strives to return to its initial position after being cut through. This increases the force to be applied by the surgeon, and to a considerable extent as the penetration depth increases. The problem is further aggravated when the surgeon operates in a small area, for example in the face or skull region.

### BRIEF SUMMARY OF THE INVENTION

Aspects of the present invention are directed to a self-tapping bone screw in which the screwing-in forces are reduced without the secure and exact-fitting seating of the screw and hence its function being adversely affected.

According to a first aspect of the present invention, the self-tapping bone screw has a screw shank, which has a front tip, a cutting region, an intermediate region and a rear head region. A thread extends, in a threaded portion of the screw shank, over a transition region comprising mutually adjoining parts of at least the cutting region and the intermediate region. An outside diameter and a root diameter of the screw shank are defined by the thread in the threaded region. In the transition region the root diameter of the screw shank in the cutting region is greater than the root diameter of the screw shank in the intermediate region. Furthermore, in the transition region the outside diameter of the screw shank is constant.

The root diameter at the transition from the cutting region to the intermediate region may be stepped, for example in the shape of a single step or a plurality of steps. In some variants of the proposed bone screw, the root of the cutting region has a convex shape. In other variants, the root of the cutting region is stepped. Mixtures of a convex and stepped contour are also conceivable. Regardless of that, the root diameter of the screw shank in the intermediate region may be constant or else vary. The outside diameter of the screw shank in the threaded portion may be constant in the intermediate region. In some realizations of the bone screw, the thread extends continuously over the cutting region and the intermediate region (and possibly also into the head region). The thread may have a constant thread pitch.

The thread may be designed in the cutting region as a trapezoidal thread, at least in regions. In the intermediate region, the thread may be constituted as a triangular thread. In certain realizations of the bone screw, the thread runs out at the tip. Regardless of this, the tip may be designed as a centring tip. For example, the tip may be designed in a stepped or rounded manner.

According to one variant of the bone screw, the latter has a groove, extending over at least the tip and the cutting region, for removing cut material. Two, three or more such grooves may also be provided.

The head region of the bone screw may have a thread. In other realizations, the head region is thread-free. The outside diameter of the head region may be greater than the outside diameter of the intermediate region. The root diameter of the head region may also be greater than the root diameter of the intermediate region.

According to one alternative, the bone screw has a thread extending continuously from the tip up to the head region. In a variant of this realization, the head region has a root diameter or outside diameter which is enlarged in each case in relation to the intermediate region. In another variant, the root diameter and outside diameter in the head region and intermediate region are constant.

In one variant, the bone screw has a tip-side threaded portion and a head-side threaded portion. The threaded portions are separated from one another by a thread-free part of the intermediate region. The threads in the two threaded portions may run synchronously with one another. The head region with the head-side threaded portion may have a greater root diameter and outside diameter than the intermediate region.

One realization of the bone screw discussed here is intended for use as a locking screw for an implant such as a bone plate. The bone screw may also be used as a compression screw either together with an implant (such as a bone plate) or without an implant. The compression screw may, for example, be utilized for compressing the bone to the implant, in which case the bone screw may be realized with a thread-free head. Realizations of the bone screw with a threaded head may be utilized in locking scenarios to lock the screw head to the plate. To this end, a plate hole receiving the bone screw may comprise a thread that is complementary to the thread on the head of the bone screw.

Furthermore, according to a further aspect a method for manufacturing a self-tapping bone screw is proposed. The bone screw has a screw shank, which has a front tip, a cutting region, an intermediate region and a rear head region. A thread extends, in a threaded portion of the screw shank, over a transition region comprising mutually adjoining parts of at least the cutting region and the intermediate region. An outside diameter and a root diameter of the screw shank are defined by the thread in the threaded region. The method comprises the step of guiding a milling tool for producing the thread in the threaded portion in such a way that the thread teeth are cut less deeply in the cutting region than in the intermediate region. In this way, in the transition region the root diameter of the screw shank in the cutting region is greater than the root diameter of the screw shank in the intermediate region. In addition, the outside diameter of the screw shank is constant.

### BRIEF DESCRIPTION OF THE DRAWINGS

Further aspects and advantages of the invention will become apparent from the following description of preferred embodiments and from the figures, in which:

FIG. 1 shows a side view of a first embodiment of a bone screw;

FIG. 2 shows a side view of the bone screw from FIG. 1 rotated by 90°;

FIG. 3 shows an enlarged side view of a front portion of the bone screw from FIG. 1;

FIG. 4 shows an enlarged side view of a front portion of the bone screw from FIG. 2;

FIG. 5 shows a top view of the tip of the bone screw from FIG. 1;

FIG. 6 shows a side view of a front portion of a screw blank;

FIG. 7 shows a section through a front portion of the bone screw from FIG. 1;

FIG. 8 shows a second embodiment of a bone screw having a head region which is altered in relation to the screw from FIG. 1;

FIG. 9 shows a third embodiment of a bone screw having a head region which is altered in relation to the screws from FIGS. 1 and 8;

FIG. 10 shows a fourth embodiment of a bone screw having a head region which is again altered in relation to the previous examples;

FIG. 11 shows a top view of the tip of the bone screw from FIG. 10;

FIG. 12 shows a section through the cutting region of the bone screw from FIG. 10;

FIG. 13 shows a sectional view of a rear part of the bone screw from FIG. 10; and

FIG. 14 shows a top view of the head region of the bone screw from FIG. 10.

### DETAILED DESCRIPTION

Several embodiments of a bone screw are explained below. In different views of one and the same embodiment, the same reference symbols are used for identical elements.

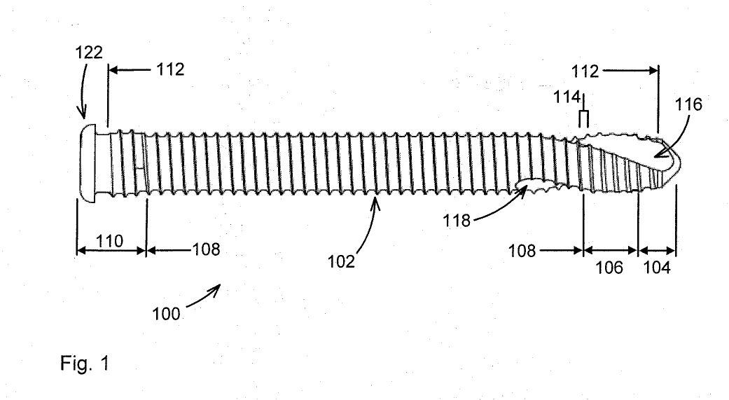

Firstly, with reference to FIGS. 1-7, a first embodiment of a bone screw 100 which can be provided for example as a locking screw for use in osteosynthesis in the face/skull region is explained. FIG. 1 shows a side view of the bone screw 100, which is designed as a self-tapping screw. The bone screw 100 has a screw shank 102 with a front tip 104, a cutting region 106, an intermediate region 108 and a head region 110. A thread extends over a threaded portion 112 which extends, in this embodiment, continuously from the tip 104 right up into the head region 110. In the front region of the screw shank 102, two helically wound grooves 116 and 118 for removing cut material are provided.

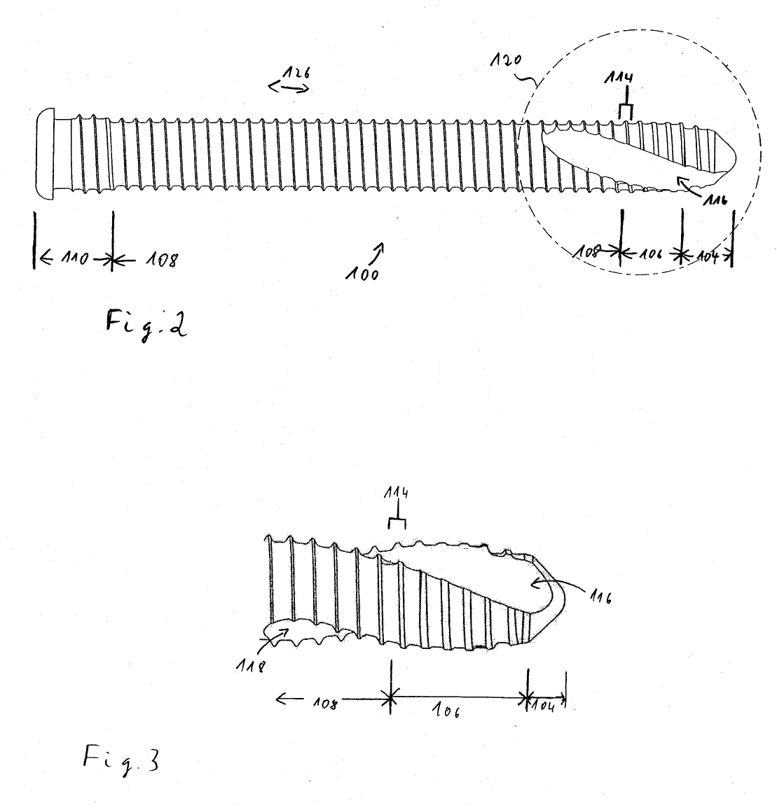

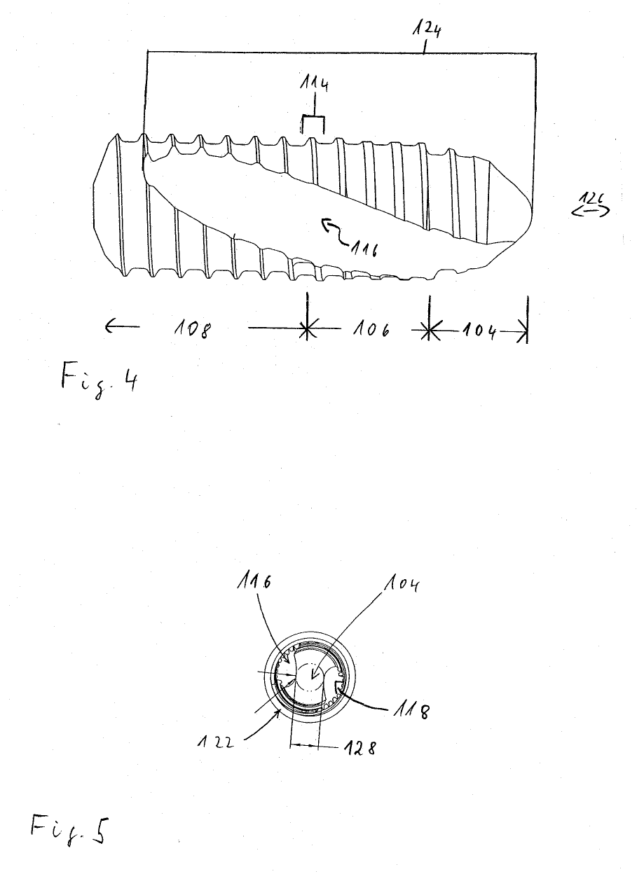

FIG. 2 shows the bone screw 100 in a view rotated by a quarter turn about the longitudinal axis. A detail of the side views of the screw 100 from FIGS. 1 and 2 (see in FIG. 2 the detail denoted by the circle 120) is illustrated in enlarged manner in FIGS. 3 and 4, respectively. It can be seen, for example, from FIG. 4 that the groove 116 extends over the tip 104 and the cutting region 106 into the intermediate region 108 and runs out there. Instead of two grooves, it is also possible for only one groove or several grooves, for example 3 or 4 grooves, to be provided. FIG. 5 shows a view, from the front, of the tip 104 of the screw 100. The grooves 116 and 118 and also the head part 122 (cf. FIG. 1) of the head region 110 can be seen.

The cutting region of the self-tapping screw 100 comprises that tip-side and threaded region 106 of the screw 100 which cuts the mating thread into the bone material. This is the region in which the thread reaches and maintains its greatest outside diameter, disregarding the fact that the outside diameter and/or root diameter are optionally further increased in the head region 110. At the head side, the cutting region 106 ends at the location at which both the greatest outside diameter and the greatest root diameter are reached and the root diameter (or the outside diameter, or both diameters) decreases in the direction of the intermediate region 108.

In the case of the screw 100, the root diameter of the cutting region 106 is increased in relation to that of the intermediate region 108. Generally, it is the case that, if only the root diameter is considered, the tip-side portion of the screw 100 can have, for example, a crowned shape. For this purpose, the root diameter in the cutting region 106 can vary, for example, in the shape of a convex curve, while in the adjoining part of the intermediate region (and possibly also the tip) it is constant. In the example of the bone screw 100, the root diameter in the region of the cutting region 106 is less crowned, but rather constant. Intermediate shapes between a crowned shape and a constant, enlarged root diameter are possible, for example a root diameter with a plurality of steps in the cutting region.

A transition region 114 is defined between the cutting region 106 and the intermediate region 108 as a result of the root diameter of the cutting region 106 merging into the root diameter of the intermediate region 108 here. As is evident from the figures, the intermediate region 108 has itself a constant root diameter. The outside diameter of the screw shank 102, when seen from the tip, reaches its greatest value in the cutting region 106 and is constant in the further course in the cutting region 106 and the intermediate region 108.

A screw blank 200, from which the screw 100 is machined, is shown schematically in FIG. 6. The centring tip 104 is produced from a region 202, the cutting region 106 from a region 204 and the intermediate region 108 from a region 206. The region 202 comprises the rounded segment 208 and the two trapezoidal segments or spherical caps 210 and 212, each having different opening angles, from which the stepped shape of the subsequent centring tip 104 results. The segment 210 remains thread-free, and the threaded portion 112 begins at the segment 212. Further segments 214 and 216 are cylindrically shaped. The root diameter which is thickened in the cutting region 106 in relation to the intermediate region 108 is formed only in the course of the thread milling.

In other embodiments, instead of the stepped tip shown in FIG. 6, continuous shapes may also be used. Generally, the tip is advantageously provided as a centring tip with the thread running out.

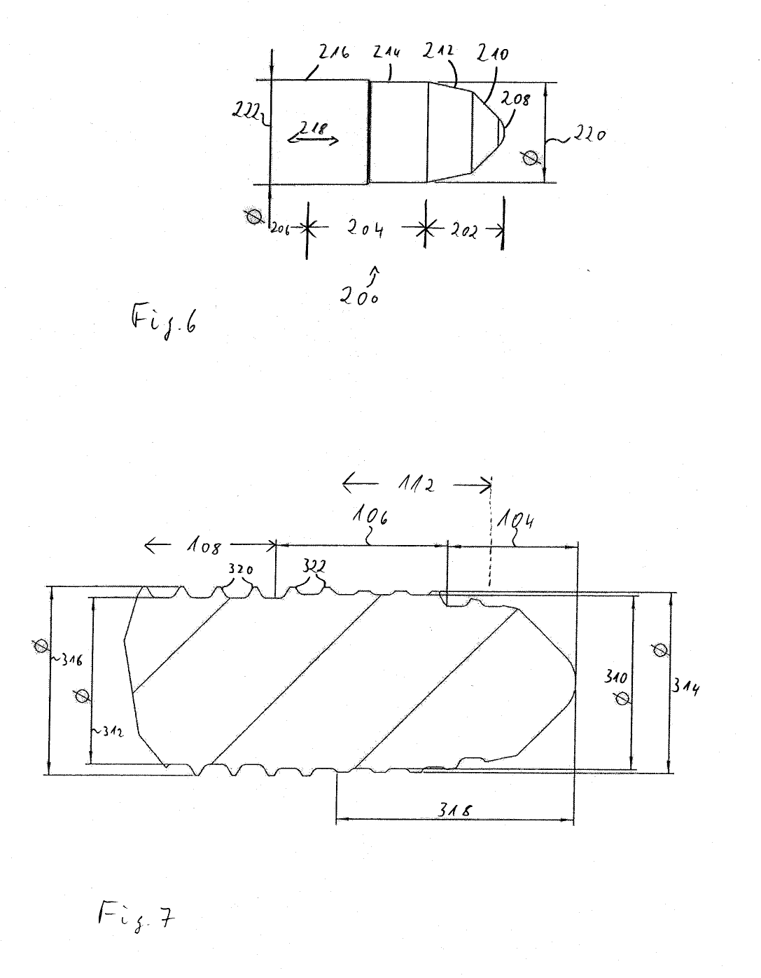

FIG. 7 is a cross-section through the part of the bone screw 100 shown in FIG. 4, with the tip 104 which opens at a right angle, the cutting region 106 and the tip-side part of the intermediate region 108.

The greatest root diameter 310 of the threaded region 112 is reached in the cutting region 106. Over the cutting region 106, the root diameter is constant. In the transition region 114 between the cutting region 106 and the intermediate region 108, the root diameter decreases to a smaller value 312, which is maintained over the intermediate region 108. The outside diameter increases from the tip 104 via a value 314 at the transition to the cutting region 106 and reaches its maximum value 316 in the cutting region 106. The outside diameter is constant with the value 316 in the transition region 114 and in the intermediate region 108.

As can be seen from FIG. 7, in the case of the bone screw 100 described by way of example here, the transition from the enlarged root diameter 310 in the cutting region 106 to the smaller root diameter 312 in the intermediate region 108 takes place in a stepped manner, that is to say the transition takes place in the region 114 in the shape of a single step. In other embodiments of the bone screw, the stepping can instead be carried out in the form of a plurality of steps, or the root diameter is reduced in the transition region, for example, in the shape of a convex curve. Mixtures of stepped and continuous reduction of the root diameter are also possible.

In the case of the single-step transition, shown in FIG. 7, from the constant root diameter 310 in the cutting region 106 to the constant root diameter 312 in the intermediate region 108, the transition region 114 is identical to the boundary between the cutting region 106 and the intermediate region 108. In other embodiments, in which the root diameter between the cutting and intermediate regions decreases in a plurality of steps and/or continuously, the transition region is accordingly more extensive.

In the example of the bone screw 100, the root diameter 312 in the intermediate region is constant. Generally, it is not absolutely necessary for the root diameter in the intermediate region to be constant. However, the root diameter in the intermediate region should be less than the root diameter in the cutting region. Furthermore, in the embodiment of the bone screw 100 shown in FIGS. 1-7, the root diameter 310 in the cutting region 106 is constant. In other embodiments, the root diameter in the cutting region may vary, and may describe, for example, a convex curve as a whole or piece by piece, for example at the transition from the tip to the cutting region (in FIG. 7 the transition of the root diameter from the tip 104 to the cutting region 106 takes place in a stepped manner).

Since the root diameter 310 in the cutting region 106 is greater than the root diameter in the intermediate region 108, a larger hole is cut by the cutting region 106 than that which corresponds to the root diameter 312 in the intermediate region 108. As a result, with respect to the bone which has been cut through, the bearing surface of the screw 100 and the penetration depth of the thread teeth 320 are reduced in the intermediate region 108. This leads to a reduction of the screwing-in forces of the screw 100 into the bone. In order that the screw 100 after being screwed in does not lie loosely, in the intermediate region 108, in the mating thread cut by the cutting region 106, the cut geometry should correspond. That is to say the thread in the cutting region 106 and the intermediate region 108 should be designed continuously and with a constant thread pitch. If, alternatively, a plurality of threaded portions (separated by thread-free portions) are provided in the cutting region and in the intermediate region, these should correspond to one another, i.e. the threads should be synchronous with one another.

As can be seen in particular in FIGS. 3, 4 and 7, the thread profile changes at the transition from the intermediate region 108 to the cutting region 106. While in the cutting region 106 (and in the threaded part running out in the tip 104) the thread is shaped trapezoidally with a comparatively large surface of the teeth (outer surface of the screw), cf. the thread teeth 322 in FIG. 7, the thread shape in the intermediate region 108 corresponds to a trapezoidal thread with a comparatively smaller surface of the teeth (more acute thread teeth), cf. the thread teeth 320. In other embodiments, a triangular thread may be present in the intermediate region. Regardless of this, the edges of the screw 100 are generally cut off, so that the thread teeth 320 are also rounded to a certain extent.

The trapezoidal cross-section of the thread turns in the cutting region 106 serves in particular for cutting through the bone material on screwing in, while the cutting function of the triangular thread in the intermediate region 108 is less important. In the intermediate region 108, the thread is intended in particular to fit into the thread turns which have already been cut, without the screwing-in resistance significantly increasing as a result.

In the case of a method for producing the screw 100 from the screw blank 200, a milling tool can be used to produce the thread. In this method, the milling tool can be guided, for example, over a convex curve or a stepping with one or more steps. The thread teeth are thereby cut less deeply in the cutting region 106 than in the intermediate region 108, thus resulting in the enlarged root diameter 310 in the cutting region 106 in relation to the root diameter 312 of the intermediate region 316.

Examples of specific dimensions of the bone screw 100 are given below. Frequently, valid ranges of values are specified with a lower and upper value in each case; from the combination of the lower values, a concrete embodiment of a smaller screw results, while the combination of the upper values results in a concrete embodiment of a larger screw. However, examples which lie outside the specified ranges of values are also readily conceivable; the general dimensions of bone screws, in particular locking screws, are known to a person skilled in the art. What is important in the case of the numerical values specified here are not only the absolute values but also the relationship of the values of the various dimensions to one another.

In general, it is the case that a typical effective diameter of the bone screw 100 may lie, for example, between 2 millimetres (mm) and 8.0 mm, preferably between 2.7 mm and 5.0 mm; smaller or larger effective diameters are likewise possible, but the following ranges of values relate to screws having the specified effective diameters. The tolerance of the dimensions specified by way of example lies typically in the region of 0.1 mm.

Owing to the lack of thread, the circumstances for the screw tip 104 are explained with the aid of the screw blank 200 shown in FIG. 6 for greater clarity. The segment 210 adjoining the rounded tip segment 208 may form a truncated cone with an opening angle of 90°, that is to say the surface lines of the truncated cone form an angle of 45° with respect to the screw (blank) axis 218. More acute or more obtuse opening angles are likewise possible; however, the self-centring properties of the screw should preferably be retained. The truncated cone formed by the adjoining segment 212 may have, for example, an opening angle of 24°, i.e. the surface lines form an angle of 12° with respect to the screw axis 218.

The cylindrical segment 214 may have a diameter 220, for example, in the range of 4.9 mm to 2.8 mm. The adjoining blank shank 216 may have, for example, a diameter 222 of 5.1 mm to 3.0 mm. A length of the segments 208, 210, 212 and 214 along the screw axis 218 may lie, for example, in the range of 6.7 mm to 4.5 mm. A length only of the segments 208, 210 and 212 of, for example, 3.76 mm to 2.59 mm could then result, and a length only of the segments 208 and 210 of 1.6 mm to 0.55 mm could result.

Referring to FIG. 7, the nominal diameter 314 at the tip-side part of the cutting region 106 is, for example, 4.9 mm to 2.8 mm, while the nominal diameter 316 at the head-side part of the cutting region 106 is, for example, 5.1 mm to 3.0 mm. The root diameter 310 in the cutting region 106 may assume, for example, a value in the range from 4.7 mm to 2.7 mm. The root diameter 312 in the intermediate region 108 has, in contrast, a smaller value in the range from 4.5 mm to 2.5 mm.

A length of the cutting region 106 along the screw axis 218 (FIG. 6) may lie, for example, between 4.7 mm and 2.0 mm. Accordingly, a length of the tip 104 may lie between 3.5 mm and 2.7 mm. The length 318 in FIG. 7 indicates the distance from the tip of the screw up to the point from which the maximum nominal or outside diameter of the screw 100 is reached. The length 318 may have, for example, a value between 6.7 mm to 4.5 mm.

The flank angle of the trapezoidal thread in the cutting region 106 may be, for example, 45°.

The groove 116 shown in particular in FIG. 4 may be formed in a manner running out to an outside diameter of 4.7 mm to 2.0 mm. The groove pitch of the grooves 116 and 118 may be typically 40 mm. The length 124 of the groove along the screw axis 126 (cf. FIGS. 2 and 4) may be between 20 mm to 2 mm and preferably between 12.5 mm to 6.0 mm. However, the length 124 of the groove 116 may be chosen to be substantially constant at 12.5 mm, almost regardless of the overall length of the screw. Only in the case of particularly short screws, for example with a length of less than 20 mm, can a shorter length 124 of the groove along the screw axis, for example with a value of 6.2 mm, be provided.

The edges of the grooves 116 and 118 are designed sharp but burr-free. The grooves 116 and 118 are offset by 180° with a tolerance of, for example, 1°. Referring to FIG. 5, the grooves 116 and 118 have at the tip 104 a spacing 128 of 2 mm, but at least 0.8 mm.

FIG. 8 shows a further embodiment of a bone screw 400. The latter differs from the bone screw 100 of the previous figures in the design of the head region. While in the case of the head region 110 of the screw 100 (cf. FIG. 1) both the root diameter and the outside diameter are enlarged in comparison with the corresponding diameters of the intermediate region 108, the head region 402 of the screw 400 has both the same root diameter and the same outside diameter as the intermediate region 404. Since the threaded portion 406 usable for screwing in and optionally locking extends right up to the head part 408 in the case of the screw 400, the screw shank 410 of the screw 400 can be designed shorter overall than the screw shank 102 of the screw 100.

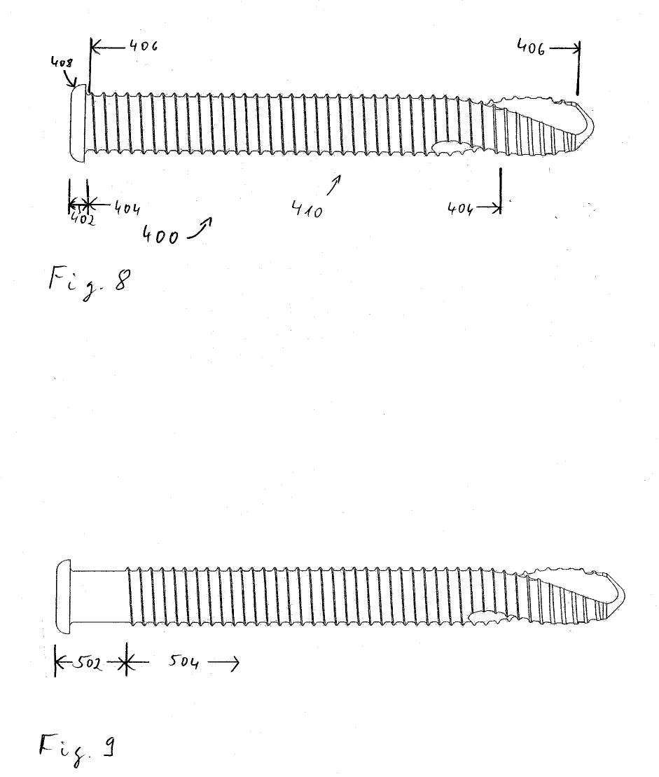

FIG. 9 shows a further embodiment 500 of a bone screw, in which the head region 502 is designed differently again than in the screws 100 and 400. The head region 502 is designed in particular thread-free. The diameter of the head region 502 corresponds to the root diameter of the intermediate region 504.

FIG. 10 shows a further embodiment of a bone screw 600. As the preceding embodiments, the screw 600 also has in the cutting region 602 an enlarged root diameter in comparison with the intermediate region 604. The outside diameter of the screw is constant in the cutting region and in the intermediate region. The obtuse screw tip 606 has an opening angle of 90°. A head region 608 has an enlarged head part 610 with its own thread.

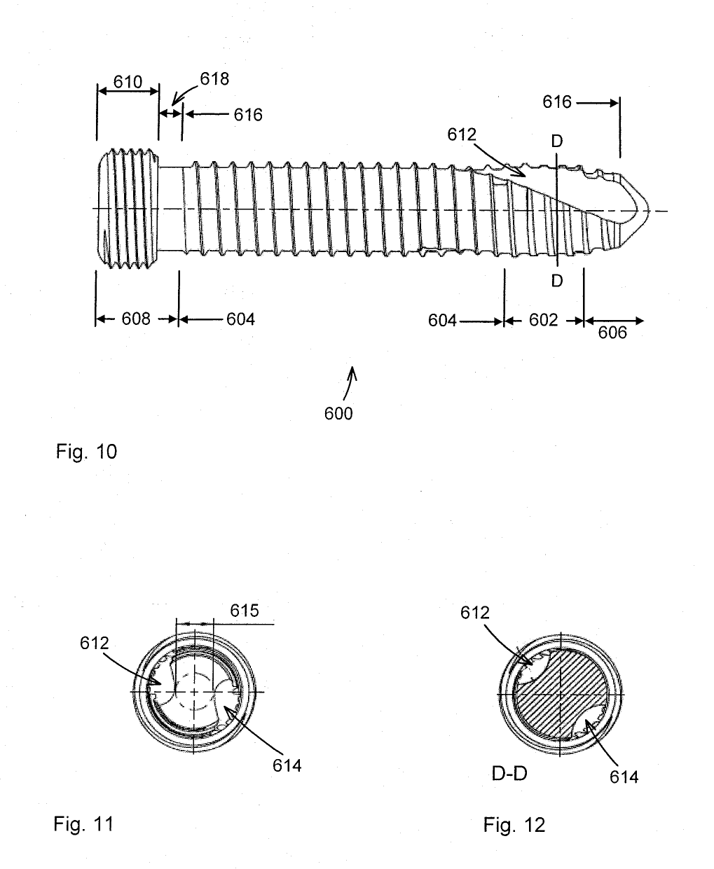

FIG. 11 shows the screw 600 from the front and FIG. 12 shows a section through the screw 600 along the line D-D in the cutting region 602. There can be seen two tapering grooves 612, 614 which can be configured in the same way as for the grooves 116, 118 of the screw 100. The grooves 612 and 614 are offset by 180°. Furthermore, the thread of the head part 610 can be seen.

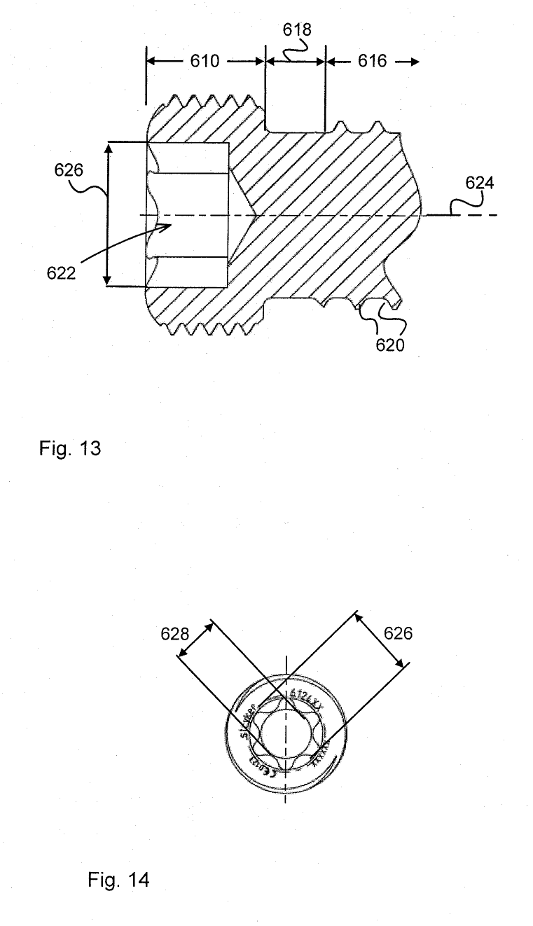

FIG. 13 is a sectional view of the rear part of the screw 600. As can be seen from the two FIGS. 10 and 13, the front threaded portion 616 in the intermediate region 604 ends at the head region 608, which thus has a thread-free portion 618. The head part 610 has a diameter significantly enlarged in relation to the intermediate region 604, that is to say both the root diameter and the outside diameter of the threaded head part 610 are greater than the corresponding diameter of the intermediate region 604. The threads in the threaded portion 616 and in the head part 610 run synchronously with one another. The thread in the head part 610 may be a two-start thread.

As with the design for the example of the bone screw 100, the thread teeth 620 in the case of the screw 600 too (cf. FIG. 13) may be configured as narrow trapeziums (in particular in comparison with the thread teeth in the cutting region), thus resulting in an acute trapezoidal thread. Alternatively, the thread in the intermediate region may also be designed as a triangular thread, it being possible for the thread teeth to be cut off.

The head region 608 is configured with a recess 622 for receiving a wrench, for example as a hexalobular internal driving feature. FIG. 14 is a top view of the rear end of the screw 600 with the head region 608 and recess 622.

An overall length of the screw 600 may lie, for example, between 8 mm and 150 mm, preferably between 14 mm and 120 mm. The length of the head part 612 along the screw axis 624 may be, for example, 3.2 mm. The thread-free portion 618 may have a length of 1.3 mm. With an effective diameter of the screw 600 of 4.7 mm in the intermediate region 604, the root diameter may be 4.5 mm in the intermediate region 604 (4.7 mm in the cutting region 602) and the outside diameter may be 5.1 mm in the intermediate region 604 and the cutting region 602. The thickened head part 610 may in this case have a root diameter of 5.8 mm and an outside diameter of 6.5 mm.

The thread teeth 620 may have a spacing of 1 mm between the teeth and have an upper trapezium surface of 0.1 mm. The spacing between the bases of two thread teeth on the screw shank may be approximately 0.323 mm.

The outer diameter 626 of the hexalobular internal driving feature 622 may be, for example, 3.95 mm and the innermost diameter 628 of the hexalobular internal driving feature 622 may be 2.85 mm, in each case with a tolerance of a few hundredths of a millimetre.

All of the bone screws described here may be used as compression screws or locking screws for an implant. While the front part in the case of the bone screws 100, 400, 500 and 600 is configured in each case in the same way, in particular with respect to the enlarged root diameter in the cutting region, the screws are adapted by means of their head region to respectively different applications, for example to different fixation elements. The material used for the bone screws illustrated by way of example here may be special steel or titanium.

The bone screws discussed above can be screwed into bones or bone fragments with a reduced screwing-in force in relation to conventional screws. The enlarged root diameter in the cutting region of the bone screw (in comparison with the root diameter of the intermediate region) has the effect that the bearing surface of the screw in the intermediate region is reduced, and at the same time the penetration depth of the thread teeth can be reduced. A threaded portion (or a plurality of separate, synchronous threaded portions) extending continuously from the cutting region up to the intermediate region and optionally to the head region ensure that the cut geometry corresponds. Thus, the intermediate region of the screw situated behind the cutting region fits exactly, on screwing in, into the cut mating thread in the bone. A constant thread pitch is advantageously provided here.

In order to ensure a secure seating of the screw also in the intermediate region, the thread shape may vary between the cutting region and the intermediate region, for example from a trapezoidal to a triangular thread, or from a more obtuse trapezoidal thread to a more acute trapezoidal thread. Other thread shapes are likewise conceivable, insofar as they ensure the functionality of a self-tapping screw. The constant outside diameter in the transition region, and preferably also along at least a substantial part of the intermediate region ensures the optimal seating of the screw here. A thread extending continuously from the cutting region over the entire intermediate region further improves the exact-fitting seating here, without the screwing-in forces substantially increasing. One or more groove may be provided in order to remove cut material without thereby impairing the functionality of the screw with respect to the reduction of the screwing-in forces with a secure seating.

Depending on the specific application, it may be expedient to provide in the head region a head part which is enlarged with respect to the root diameter and/or outside diameter in order to ensure a secure seating of the bone screw in the bone material and/or a further implant element. The correspondingly thickened head part may have its own, optionally synchronous thread, or a continuous threaded portion extends from the intermediate region into the head region.

The embodiments illustrated here represent only a few expedient embodiments of the invention. Within the scope of the invention specified by the following claims, many other embodiments besides will be conceivable by those skilled in the art.

1. A self-tapping bone screw comprising:
a screw shank having a front tip, a cutting region, an intermediate region and a rear head region;
a thread extending in a threaded portion of the screw shank over at least a transition region including mutually adjoining parts of the cutting region and the intermediate region; and
an outside diameter and a root diameter of the screw shank being defined by the thread in the threaded region,
wherein, in the transition region, the root diameter of the screw shank in the cutting region is greater than the root diameter of the screw shank in the intermediate region and the outside diameter of the screw shank is constant.

2. The bone screw according to claim 1, wherein the root diameter at the transition from the cutting region to the intermediate region is stepped.

3. The bone screw according to claim 1, wherein the root of the cutting region has a convex shape.

4. The bone screw according to claim 1, wherein the root diameter in the intermediate region is constant.

5. The bone screw according to claim 1, wherein the outside diameter in the intermediate region is constant.

6. The bone screw according to claim 1, wherein the thread extends continuously over the cutting region and the intermediate region.

7. The bone screw according to claim 1, wherein the thread in the cutting region is designed as a trapezoidal thread.

8. The bone screw according to claim 1, wherein the thread has a constant thread pitch.

9. The bone screw according to claim 1, wherein the thread runs out in the front tip.

10. The bone screw according to claim 1, wherein the front tip is a centring tip.

11. The bone screw according to claim 1, further comprising at least one groove extending over at least the tip and the cutting region for removing cut material.

12. The bone screw according to claim 1, wherein the head region has a thread.

13. The bone screw according to claim 1, wherein the outside diameter of the head region is greater than the outside diameter of the intermediate region.

14. The bone screw according to claim 1 for use as a compression screw or a locking screw for an implant.

15. A method for manufacturing a self-tapping bone screw having a screw shank including a front tip, a cutting region, an intermediate region and a rear head region, a thread extending, in a threaded portion of the screw shank, over a transition region including mutually adjoining parts of the cutting region and the intermediate region, and an outside diameter and a root diameter of the screw shank being defined by the thread in the threaded region, the method comprising the steps of:
guiding a milling tool for producing the thread in the threaded portion in such a way that the thread teeth are cut less deeply in the cutting region than in the intermediate region, so that in the transition region, the root diameter of the screw shank in the cutting region is greater than the root diameter of the screw shank in the intermediate region and the outside diameter of the screw shank is constant.
